(19) United States
(12) Patent Application Publication
(10) Pub. No.: US 2017/0360596 A1
(43) Pub. Date: Dec. 21, 2017

(54) AIRSTOP SYSTEM AND METHOD

(71) Applicants: Charles E. Knowles, II, Wichita, KS (US); Janis Knowles, Wichita, KS (US); Matthew Knowles, Vero Beach, FL (US)

(72) Inventors: Charles E. Knowles, II, Wichita, KS (US); Janis Knowles, Wichita, KS (US); Matthew Knowles, Vero Beach, FL (US)

(21) Appl. No.: 15/626,051

(22) Filed: Jun. 16, 2017

Related U.S. Application Data
(60) Provisional application No. 62/351,274, filed on Jun. 16, 2016.

(51) Int. Cl. A61F 5/56; A61M 16/00

(52) U.S. Cl. CPC A61F 5/566; A61M 16/0057

(57) ABSTRACT

A mouthpiece for inhibiting breathing through a mouth of a user-wearer is disclosed herein. The mouthpiece for inhibiting breathing through a mouth of a user-wearer includes a blocking sheet, a relief port, and a flex recess. The blocking sheet may be made of a flexible material impermeable to air, and is configured to fit within the mouth of the user-wearer between the front teeth and the lips as well as the cheek area. The mouthpiece creates improved airflow for CPAP users by restricting airflow through the mouth.

### CROSS-REFERENCE TO RELATED APPLICATION(S)

The present application is related to and claims priority to U.S. Provisional Patent Application No. 62/351,274 filed Jun. 16, 2016, which is incorporated by reference herein in its entirety.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### FIELD OF THE INVENTION

The present invention relates generally to the field of durable medical equipment of existing art and more specifically relates to Continuous Positive Airway Pressure (CPAP) equipment.

### DESCRIPTION OF RELATED ART

Individuals using Continuous Positive Airway Pressure (CPAP) treatments often have to wear a mask or headgear to prevent mouth breathing. Air leakage during treatment can diminish the effectiveness of the Continuous Positive Airway Pressure (CPAP) treatment, and in addition can cause dry mouth and a sore throat. Even individuals not using a Continuous Positive Airway Pressure (CPAP) machine can experience negative effects of breathing through their mouth, such as poor sleeping habits, gum disease, headaches, abnormal facial development and more. An efficient method for preventing mouth breathing is needed.

U.S. Pat. No. 7,676,276 to Manuel L. Karell relates to a stimulator combined with an intranasal respiratory method and device for improved breathing. The described stimulator combined with an intranasal respiratory method and device for improved breathing includes a nasal respiratory device and method adapted to provide electrical stimulation comprising an intraoral holdfast having a neuromuscular stimulator with electrodes adapted to provide electrical stimulation to a user for improving breathing and treating a respiratory disorder. The device and method affords access to ambient atmosphere or may be connected pressure devices such as CPAP.

### SUMMARY OF THE INVENTION

In view of the foregoing disadvantages inherent in the known Continuous Positive Airway Pressure (CPAP) equipment art, the present disclosure provides a novel mouthpiece for inhibiting breathing through a mouth of a user-wearer. The general purpose of the present disclosure, which will be described subsequently in greater detail, is to provide a mouthpiece for inhibiting breathing through a mouth of a user-wearer to increase the effectiveness of a Continuous Positive Airway Pressure (CPAP) machine.

A mouthpiece for inhibiting breathing through a mouth of a user-wearer is disclosed herein. The mouthpiece for inhibiting breathing through a mouth of a user-wearer includes a blocking sheet, a relief port, and a flex recess. The blocking sheet may be made of a flexible material impermeable to air; and is configured to fit within the mouth of the user-wearer between the front teeth and the lips as well as the cheek area, further configured to block air from escaping the mouth when worn between the front teeth and the lips; and even further having a sheet thickness, a height, a width, a vertical center axis, and a horizontal center axis, all relative to the mouth of the user-wearer when worn.

The blocking sheet may include exactly twelve curved edges and is configured to assist with smooth insertion into the mouth of the user-wearer, and alternately, less than twelve curved edges are configured to assist with smooth insertion into the mouth of the user-wearer. The length of the flexible material of the blocking sheet measures at least wide enough to fit comfortably in the mouth of the user-wearer. The height of the flexible material of the blocking sheet is configured to measure at least high enough to fit comfortably in the mouth of the user-wearer. The material of the blocking sheet may be malleable and is configured to allow for easy insertion into the mouth of the user-wearer between the front teeth and the lips, extending into the cheek area.

The blocking sheet may be constructed from foam material and is configured to prevent mouth sores. The blocking sheet may be configured to flex at least enough to allow the mouth of the user-wearer to close and open comfortably. The blocking sheet may be constructed from foam; and configured to be impermeable to liquid. The blocking sheet may alternately be constructed from polyurethane configured to form to the shape of the mouth of the user-wearer; and may be configured to flex at least enough to allow the mouth of the user-wearer to close and open comfortably. The blocking sheet, which may alternately be constructed from polyurethane may be configured to be impermeable to liquid.

The relief port may be integrated into the blocking sheet, and is configured to provide a passageway through the blocking sheet; the relief port may include an aperture of between 0.015 and 0.13 square inches which may allow saliva to escape. The relief port may be at least one-half-inch from the lower edge of the blocking sheet when placed in the lower front teeth and lower lip, and alternately, at least three-fourths of an inch from the lower edge of the blocking sheet when placed in the lower front teeth and lower lip.

The flex recess may be integrated into the blocking sheet and is configured to allow the blocking sheet to flex when worn between the front teeth and the lips. The flex recess further includes an upper notch and a lower notch opposite the upper notch relative to the height of the blocking sheet; the upper notch and the lower notch positioned proximate the vertical center axis of the blocking sheet. The flex recess may have a V-shape, and alternately, a rounded shape.

According to another embodiment, a method for using the mouthpiece is also disclosed herein. The method for using the mouthpiece includes inserting a mouthpiece into a mouth of a user-wearer for improving flow from a CPAP machine, removing the mouthpiece from the mouth of the user-wearer when desired, and cleaning a mouthpiece after use.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a mouthpiece for inhibiting breathing through a mouth of a user-wearer, constructed and operative according to the teachings of the present disclosure.

FIG. 1 is a perspective view of the mouthpiece for inhibiting breathing through a mouth of a user-wearer during an ‘in-use’ condition, according to an embodiment of the disclosure.

FIG. 2 is a perspective view of the mouthpiece for inhibiting breathing through a mouth of a user-wearer, according to an embodiment of the present disclosure.

FIGS. 3A-3B are perspective views of the mouthpiece for inhibiting breathing through a mouth of a user-wearer, according to an embodiment of the present disclosure.

FIG. 4 is a perspective view of the mouthpiece for inhibiting breathing through a mouth of a user-wearer, according to an embodiment of the present disclosure.

FIG. 5 is a flow diagram illustrating a method for using the mouthpiece, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to Continuous Positive Airway Pressure (CPAP) equipment and more particularly to a mouthpiece for inhibiting breathing through a mouth of a user-wearer as used to improve the effectiveness of a Continuous Positive Airway Pressure (CPAP) machine.

Generally, AirStop is designed to prevent mouth breathing and to aid those using CPAP treatment to prevent air leaking through the mouth. This reduces the discomfort of air escaping through the mouth while sleeping and receiving CPAP treatment through a nosepiece. AirStop lowers the risk of abnormal facial and dental development, poor sleeping habits, gingivitis, gum disease, bad breath, and headaches that can be caused by mouth breathing. The mouthpiece is a thin pliable piece of material with a half-inch horizontal slit enabling saliva to pass through. AirStop is inserted between the upper and lower gum/teeth area and the upper and lower lips to prevent mouth breathing, and can be customized by the user ensuring a proper fit. The mouthpiece can also be used while awake if a person has issues with mouth breathing during the day.

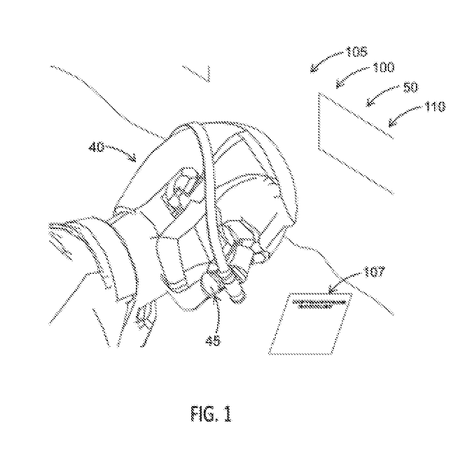

Referring now more specifically to the drawings by numerals of reference, there is shown in FIGS. 1-4, various views of a mouthpiece for inhibiting breathing through a mouth of a user-wearer 100.

FIG. 1 shows a mouthpiece for inhibiting breathing through a mouth of a user-wearer 100 during an ‘in-use’ condition 50, according to an embodiment of the present disclosure. Here, the mouthpiece for inhibiting breathing through a mouth of a user-wearer 100 may be beneficial for use by a user-wearer 40 to block airflow from a mouth 45 of a user-wearer 40. As illustrated, the mouthpiece for inhibiting breathing through a mouth 45 of a user-wearer 100 may include a blocking sheet 110, a relief port 112, and a flex recess 114. A user-wearer 40 has the mouthpiece for inhibiting breathing through the mouth 100 inserted while receiving treatment from a CPAP 35 machine during sleep.

The blocking sheet 110 may be made of a flexible material 116 impermeable to air; and is configured to fit within the mouth 45 of the user-wearer between the front teeth 60 and the lips 55 as well as the cheek area 65. The blocking sheet 100 may further be configured to block air from escaping the mouth 45 when worn between the front teeth 60 and the lips 55. The blocking sheet 100 may even further having a sheet thickness 118, and also have a height 120, a width 122, a vertical center axis, and a horizontal center axis, all relative to the mouth 45 of the user-wearer 40 when worn. The blocking sheet 110 may be constructed from foam 124 and is configured to be impermeable to liquid; may further include less than twelve curved edges 126 and is malleable to assist with smooth insertion into the mouth 45 of the user-wearer 40. The foam material 128 may be configured to prevent mouth sores, to form to the shape of the mouth 45 of the user-wearer 40, and to flex at least enough to allow the mouth 45 of the user-wearer 40 to close and open comfortably. The length 130 and height 120 of the flexible material 116 of the blocking sheet 110 may measure at least wide and high enough to fit comfortably in the mouth 45 of the user-wearer 40. The relief port 112 may be integrated into the blocking sheet 110, and is configured to provide a passageway through the blocking sheet 110, include an aperture 132 of between 0.015 and 0.13 square inches; and may be placed at least one-half-inch from the lower edge of the blocking sheet 110 when placed in the lower front teeth 60 and lower lip 70. The V-shaped flex recess 114 integrated into the blocking sheet 110 may be configured to allow the blocking sheet 110 to flex when worn between the front teeth 60 and the lips 55; include an upper notch 134 and a lower notch 136 opposite the upper notch 134 relative to the height 120 of the blocking sheet 110; the upper notch 134 and the lower notch 136 positioned proximate the vertical center axis of the blocking sheet 110.

According to one embodiment, the mouthpiece for inhibiting breathing through a mouth of a user-wearer 100 may be arranged as a kit 105. In particular, the mouthpiece for inhibiting breathing through a mouth of a user-wearer 100 may further include a set of instructions 107. The instructions 107 may detail functional relationships in relation to the structure of the mouthpiece for inhibiting breathing through a mouth of a user-wearer 100 such that the mouthpiece for inhibiting breathing through a mouth of a user-wearer 100 can be used, maintained, or the like, in a preferred manner.

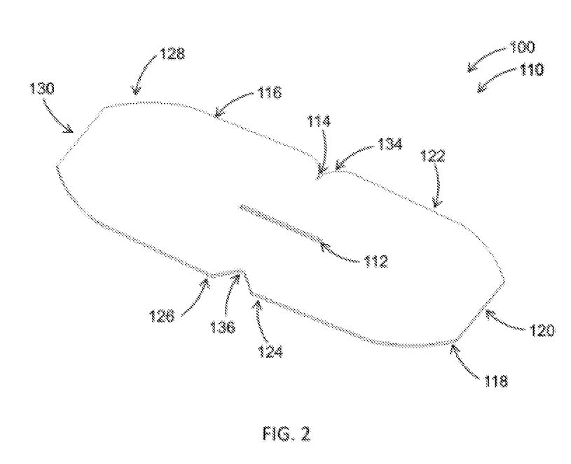

FIG. 2 shows the mouthpiece for inhibiting breathing through a mouth of a user-wearer 100, according to an embodiment of the present disclosure. As above, the mouthpiece for inhibiting breathing through a mouth of a user-wearer 100 may include a blocking sheet 110, a relief port 112, and a flex recess 114. The blocking sheet 110 may include flexible material 116 impermeable to air, and is configured to fit within the mouth 45 of the user-wearer between the front teeth 60 and the lips 55, as well as the cheek area 65. The relief port 112 may include a passageway through the blocking sheet 110, include an aperture 132 of between 0.015 and 0.13 square inches; and may be placed at least one-half-inch from the lower edge of the blocking sheet 110 when placed in the lower front teeth 60 and lower lip 70. The V-shaped flex recess 114 integrated into the blocking sheet 110 may be configured to allow the blocking sheet 110 to flex when worn between the front teeth 60 and the lips 55.

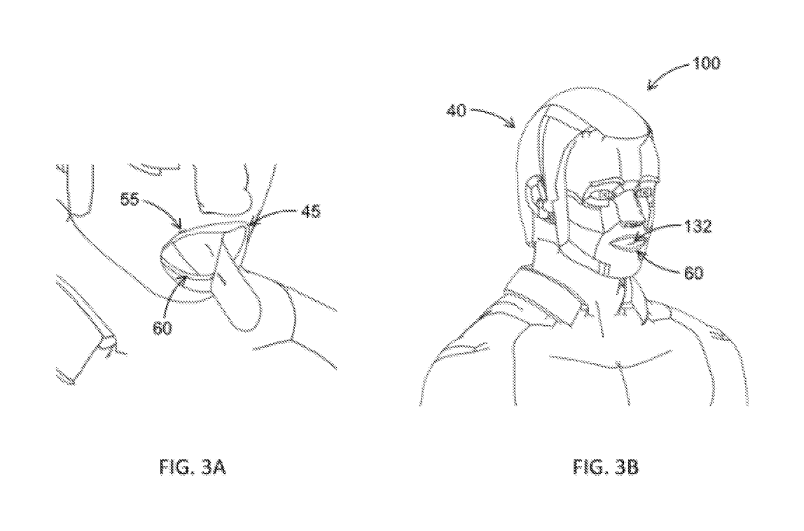

FIGS. 3A-3B are perspective views of the mouthpiece for inhibiting breathing through a mouth of a user-wearer 100, according to an embodiment of the present disclosure. The blocking sheet 110 may be constructed from foam 124 and is configured to be impermeable to liquid; may include less than twelve curved edges 126 and is malleable configured to assist with smooth insertion into the mouth 45 of the user-wearer 40. The foam material 128 may be configured to prevent mouth sores, to form to the shape of the mouth 45 of the user-wearer 40, and to flex at least enough to allow the mouth 45 of the user-wearer 40 to close and open comfortably. The length 130 and height 120 of the flexible material 116 of the blocking sheet 110 may measure at least wide and high enough to fit comfortably in the mouth 45 of the user-wearer 40. The V-shaped flex recess 114 includes an upper notch 134 and a lower notch 136 opposite the upper notch 134 relative to the height 120 of the blocking sheet 110, the upper notch 134 and the lower notch 136 positioned proximate the vertical center axis of the blocking sheet 110.

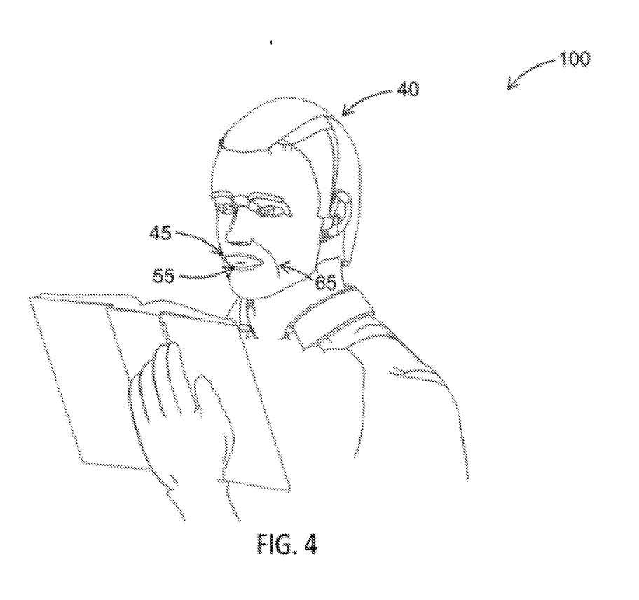

FIG. 4 is a perspective view of the mouthpiece for inhibiting breathing through a mouth of a user-wearer 100, according to an embodiment of the present disclosure. The length 130 and height 120 of the flexible material 116 of the blocking sheet 110 may measure at least wide and high enough to fit comfortably in the mouth 45 of the user-wearer 40. The relief port 112 may be integrated into the blocking sheet 110, and is configured to provide a passageway through the blocking sheet 110. The mouthpiece for inhibiting breathing through the mouth of a user-wearer 100 may be used when desired.

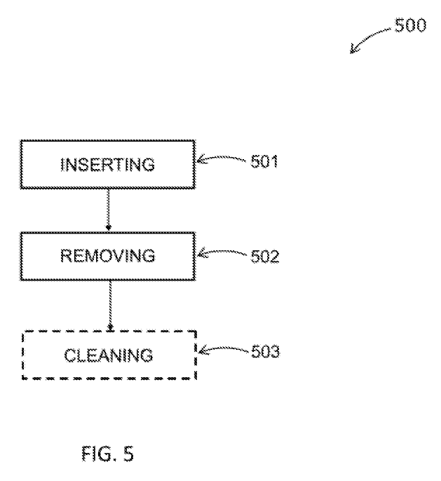

FIG. 5 is a flow diagram illustrating a method for using the mouthpiece 500, providing a mouthpiece for inhibiting breathing through a mouth of a user-wearer 100, the mouth 45 including front teeth 60 and lips 55. The mouthpiece may comprise a blocking sheet 110 made of a flexible material 116 impermeable to air. The blocking sheet 110 may be configured to fit within the mouth 45 of the user-wearer 40 between the front teeth 60 and the lips 55; and further configured to block air from escaping the mouth 45 when worn between the front teeth 60 and the lips 55. The blocking sheet 110 may have a sheet thickness 118 and a height 120, a width 122, a vertical center axis, and a horizontal center axis; all relative to the mouth 45 of the user-wearer 40 when worn. A relief port 112 may be integrated into the blocking sheet 110, the relief port 112 configured to provide a passageway through the blocking sheet 110. The relief port includes an aperture of between 0.015 and 0.13 square inches. A flex recess 114 may also be integrated into the blocking sheet 110 and configured to allow the blocking sheet 110 to flex when worn between the front teeth 60 and the lips 55. The flex recess 114 may further include an upper notch 134 and a lower notch 136 opposite the upper notch 134 relative to the height 12 of the blocking sheet 110. The upper notch 134 and the lower notch 136 are positioned proximate the vertical center axis of the blocking sheet 110, according to an embodiment of the present disclosure. In particular, the method for using the mouthpiece 500 may include one or more components or features of the mouthpiece for inhibiting breathing through a mouth of a user-wearer 100 as described above. As illustrated, the method for using the mouthpiece 500 may include the steps of: step one 501, inserting a mouthpiece into a mouth 45 of a user-wearer 40; step two 502, removing the mouthpiece from the mouth 45 of the user-wearer 40 when desired; and step three 503, cleaning the mouthpiece after use.

It should be noted that step 503 is an optional step and may not be implemented in all cases. Optional steps of method of use 500 are illustrated using dotted lines in FIG. 5 so as to distinguish them from the other steps of method of use 500. It should also be noted that the steps described in the method of use can be carried out in many different orders according to user preference. The use of “step of” should not be interpreted as “step for”, in the claims herein and is not intended to invoke the provisions of 35 U.S.C. §112(f). It should also be noted that, under appropriate circumstances, considering such issues as design preference, user preferences, marketing preferences, cost, structural requirements, available materials, technological advances, etc., other methods for using the mouthpiece, are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application. Upon reading this specification, it should be appreciated that, under appropriate circumstances, considering such issues as user preferences, design preference, structural requirements, marketing preferences, cost, available materials, technological advances, etc., other arrangements may be sufficient.

what is claimed is new and desired to be protected by Letters Patent is set forth in the appended claims:

1. A mouthpiece for inhibiting breathing through a mouth of a user-wearer, the mouth including front teeth and lips, the mouthpiece comprising:
a blocking sheet made of a flexible material impermeable to air, the blocking sheet configured to fit within the mouth of the user-wearer between the front teeth and the lips, the blocking sheet further configured to block air from escaping the mouth when worn between the front teeth and the lips, the blocking sheet having a sheet thickness, and also having a height, a width, a vertical center axis, and a horizontal center axis, all relative to the mouth of the user-wearer when worn;
a relief port integrated into the blocking sheet, the relief port configured to provide a passageway through the blocking sheet, the relief port including an aperture of between 0.015 and 0.13 square inches; and
a flex recess integrated into the blocking sheet and configured to allow the blocking sheet, to flex when worn between the front teeth and the lips, the flex recess including an upper notch and a lower notch opposite the upper notch relative to the height of the blocking sheet, the upper notch and the lower notch positioned proximate the vertical center axis of the blocking sheet.

2. The mouthpiece of claim 1, wherein the flex recess is V-shaped.

3. The mouthpiece of claim 1, wherein the flex recess is rounded.

4. The mouthpiece of claim 1, wherein the blocking sheet is constructed from foam material configured to prevent mouth sores.

5. The mouthpiece of claim 1, wherein the blocking sheet is constructed from polyurethane configured to form to the shape of the mouth of the user-wearer.

6. The mouthpiece of claim 1, wherein the blocking sheet includes exactly twelve curved edges configured to assist with smooth insertion into the mouth of the user-wearer.

7. The mouthpiece of claim 1, wherein the length of the flexible material of the blocking sheet measures at least wide enough to fit comfortably in the mouth of the user-wearer.

8. The mouthpiece of claim 1, wherein the height of the flexible material of the blocking sheet is configured to measure at least high enough to fit comfortably in the mouth of the user-wearer.

9. The mouthpiece of claim 1, wherein the relief port is at least one-half-inch from the lower edge of the blocking sheet when placed in the lower front teeth and lower lip.

10. The mouthpiece of claim 1, wherein the relief port is at least three-fourths of an inch from the lower edge of the blocking sheet when placed in the lower front teeth and lower lip.

11. The mouthpiece of claim 1, wherein the foam of the blocking sheet is configured to flex at least enough to allow the mouth of the user-wearer to close and open comfortably.

12. The mouthpiece of claim 1, wherein the polyurethane of the blocking sheet is configured to flex at least enough to allow the mouth of the user-wearer to close and open comfortably.

13. The mouthpiece of claim 5, wherein the blocking sheet constructed from polyurethane is configured to be impermeable to liquid.

14. The mouthpiece of claim 4, wherein the blocking sheet constructed from foam is configured to be impermeable to liquid.

15. The mouthpiece of claim 6, wherein the blocking sheet includes less than twelve curved edges configured to assist with smooth insertion into the mouth of the user-wearer.

16. The mouthpiece of claim 1, wherein the blocking sheet is malleable configured to allow for easy insertion.

17. A mouthpiece for inhibiting breathing through a mouth of a user-wearer, the mouth including front teeth and lips, the mouthpiece comprising:
a blocking sheet made of a flexible material impermeable to air, the blocking sheet configured to fit within the mouth of the user-wearer between the front teeth and the lips, the blocking sheet further configured to block air from escaping the mouth when worn between the front teeth and the lips, the blocking sheet having a sheet thickness, and also having a height, a width, a vertical center axis, and a horizontal center axis, all relative to the mouth of the user-wearer when worn;
a relief port integrated into the blocking sheet, the relief port configured to provide a passageway through the blocking sheet, the relief port including an aperture of between 0.015 and 0.13 square inches; and
a flex recess integrated into the blocking sheet and configured to allow the blocking sheet, to flex when worn between the front teeth and the lips, the flex recess including an upper notch and a lower notch opposite the upper notch relative to the height of the blocking sheet, the upper notch and the lower notch positioned proximate the vertical center axis of the blocking sheet;
wherein the blocking sheet constructed from foam is configured to be impermeable to liquid;
wherein the blocking sheet includes less than twelve curved edges and is malleable configured to assist with smooth insertion into the mouth of the user-wearer;
wherein the blocking sheet is constructed from foam material configured to prevent mouth sores, to form to the shape of the mouth of the user-wearer, to flex at least enough to allow the mouth of the user-wearer to close and open comfortably; and
wherein the length and height of the flexible material of the blocking sheet measures at least wide and high enough to fit comfortably in the mouth of the user-wearer;
wherein the relief port is at least one-half-inch from the lower edge of the blocking sheet when placed in the lower front teeth and lower lip; and
wherein the flex recess is V-shaped.

18. The mouthpiece of claim 17, further comprising set of instructions; and
wherein the mouthpiece is arranged as a kit.

19. A method for using the mouthpiece, providing a mouthpiece for inhibiting breathing through a mouth of a user-wearer, the mouth including front teeth and lips, the mouthpiece comprising:
a blocking sheet made of a flexible material impermeable to air, the blocking sheet configured to fit within the mouth of the user-wearer between the front teeth and the lips, the blocking sheet further configured to block air from escaping the mouth when worn between the front teeth and the lips, the blocking sheet having a sheet thickness, and also having a height, a width, a vertical center axis, and a horizontal center axis, all relative to the mouth of the user-wearer when worn;
a relief port integrated into the blocking sheet, the relief port configured to provide a passageway through the blocking sheet, the relief port including an aperture of between 0.015 and 0.13 square inches; and
a flex recess integrated into the blocking sheet and configured to allow the blocking sheet, to flex when worn between the front teeth and the lips, the flex recess including an upper notch and a lower notch opposite the upper notch relative to the height of the blocking sheet, the upper notch and the lower notch positioned proximate the vertical center axis of the blocking sheet:
the method comprising the steps of:
inserting a mouthpiece into a mouth of a user-wearer for improving flow from a CPAP machine; and
removing the mouthpiece from the mouth of the user-wearer when desired.

20. The method of claim 19, further comprising the step of:
cleaning a mouthpiece after use.
